(19) United States
(12) Patent Application Publication
(10) Pub. No.: US 2023/0146057 A1
(43) Pub. Date: May 11, 2023

(54) SYSTEMS AND METHODS FOR SUPPORTING MEDICAL PROCEDURES

(71) Applicant: Replicare LLC, San Antonio, TX (US)

(72) Inventors: Stephen Trey Moore, San Antonio, TX (US); Rob Wayne Wilburn, San Antonio, TX (US)

(21) Appl. No.: 18/093,739

(22) Filed: Jan. 5, 2023

Related U.S. Application Data
(63) Continuation of application No. PCT/US2021/040899, filed on Jul. 8, 2021.
(60) Provisional application No. 63/049,479, filed on Jul. 8, 2020.

(51) Int. Cl. A61B 90/00; G16H 80/00

(52) U.S. Cl. CPC A61B 90/37; A61B 90/361; G16H 80/00

(57) ABSTRACT

Systems and methods are described herein for supporting medical procedures by allowing representatives access to real-time information concerning an ongoing medical procedure and providing for communications concerning how to make use of medical devices during such medical procedures. Such systems and methods may, for example, provide live streaming and interactive capabilities for communication between medical personnel and a medical device representative.

### CROSS REFERENCE TO RELATED APPLICATIONS

This application claims priority to U.S. Provisional Patent Application Ser. No. 63/049,479 filed Jul. 8, 2020.

### FIELD

This application relates generally to the field of computer systems and associated devices to assist medical personnel during medical procedures.

### BACKGROUND

This section is intended to provide a background or context to the invention that is recited in the claims. The description herein may include concepts that could be pursued but are not necessarily ones that have been previously conceived or pursued. Therefore, unless otherwise indicated herein, what is described in this section is not prior art to the description and claims in this application and is not admitted to be prior art by inclusion in this section.

A wide range of different medical devices, such as new instruments and robotic machines and implantable devices, are now becoming available for use by medical professionals. For example, new medical devices for various types of surgical procedures are rapidly being introduced. Moreover, new protocols and means for using existing medical devices are constantly being developed. While providing powerful and new capabilities, such advances present difficult operational challenges for medical professionals. It may be difficult for a medical professional to master the use of all such medical devices and protocols. For this reason, it would be useful for a medical professional to have the ability to interact with a representative of a medical device during the course of a medical procedure, such as a surgical procedure. However, this provides a number of complications. For example, the presence of additional persons in a surgical room is costly and provides increased opportunities for transmission of microbes, such as the novel coronavirus that causes COVID-19, for example, into the surgical setting. Accordingly, there is a need for systems and methods allowing a medical device representative or specialized technician to support a surgeon doing a surgical procedure where the surgeon is using that representative's equipment in the procedure without requiring the representative to be physically present in the operating room. Systems and methods described herein enable such a representative to see in real-time what is happening during a surgery and easily communicate with the surgeon as if the representative was in the room, thereby reducing the risk of transmission of diseases such as COVID-19, for example. The medical device representative may, for example, be in an adjoining area to a surgical center or surgery room, or at some other location that is nearby or remote from the medical facility.

### SUMMARY

A system for supporting a medical procedure involving a medical device may include one or more communication devices including a microphone, a speaker, a video camera, and a display, the one or more communication devices configured to collect audio and visual data of an ongoing medical procedure within an operating room and to provide the audio and visual data to a representative device. The representative device may be configured to receive the audio and visual data, play an audible representation of the audio data, and display a visual representation of the visual data. The one or more communication devices may be further configured to receive support audio and visual data regarding the use of the medical device from the representative device, play an audible representation of the support audio data, and display a visual representation of the support visual data in real-time during the medical procedure.

A system for supporting a medical procedure involving a medical device may include a representative device configured to receive audio and visual data of an ongoing medical procedure from one or more communication devices configured to collect the audio and visual data. The representative device may be further configured to play an audible representation of an audible portion of the audio and visual data and display a visual representation of a visual portion of the audio and visual data. The representative device may be further configured to transmit support audio and visual data regarding the use of the medical device to the one or more communication devices.

A method for supporting a medical procedure involving a patient and a medical device may include establishing a communication link between one or more communication devices, the one or more communication devices being configured for communicating audio and visual data for a medical procedure performed at a first location, and one or more representative devices located at one or more other locations; collecting the audio and visual data; providing the audio and visual data to the one or more representative devices; playing an audible and/or visual representation of the audio and visual data on the one or more representative devices; generating support audio and visual data on the one or more representative devices based on the audible and/or visual representation of the audio and visual data, the support audio and visual data comprising information regarding use of the medical device; and providing the support audio and visual data to the one or more communication devices in real-time during the medical procedure.

A system for demonstrating how a medical device may be used in a medical procedure, the system including one or more communication devices including a microphone, a speaker, a video camera, and a display, the one or more communication devices configured to collect audio and visual data for how said medical device may be used in a demonstration of a medical procedure and to provide said audio and visual data to a representative device; the representative device being configured to receive said audio and visual data, play an audible representation of said audio data, and display a visual representation of said visual data; the one or more communication devices being further configured to receive support audio and visual data regarding how the medical device may be used from the representative device, play an audible representation of said support audio data, and display a visual representation of said support visual data in real-time during the demonstration of the medical procedure.

A method for providing a demonstration for how a medical device may be used in a medical procedure, the method including establishing a communication link between one or more communication devices, the one or more communication devices being configured for communicating audio and visual data for how said medical device may be used in a demonstration of a medical procedure at a first location, and one or more representative devices located at one or more other locations; collecting said audio and visual data; providing said audio and visual data to the one or more representative devices; playing an audible and/or visual representation of said audio and visual data on said one or more representative devices; generating support audio and visual data on said one or more representative devices based on the played audible and/or visual representation of said audio and visual data, said support audio and visual data comprising information regarding use of the medical device during the demonstration; and providing the support audio and visual data to the one or more communication devices in real-time during said demonstration.

### BRIEF DESCRIPTION OF THE DRAWINGS

FIG. 1 is a schematic diagram of a surgical operating room within a hospital and a communication system for providing audio and video communications between personnel in the operating room and personnel outside the operating room during a surgical procedure.

FIG. 2 is a flowchart of an embodiment of a method for supporting a medical procedure by providing a medical device representative access to real-time audio and video information concerning the medical procedure and providing support audio and visual communication from the medical device representative to the medical personnel conducting the medical procedure concerning how to use the relevant medical devices.

### DETAILED DESCRIPTION

As used herein, the following terms should be understood to have the indicated meanings:

When an item is introduced by “a” or “an,” it should be understood to mean one or more of that item.

“Communication” means the transmission of one or more signals from one point to another point. Communication between two objects may be direct, or it may be indirect through one or more intermediate objects. Communication in and among computers, I/O devices and network devices may be accomplished using a variety of protocols. Protocols may include, for example, signaling, error detection and correction, data formatting and address mapping. For example, protocols may be provided according to the seven-layer Open Systems Interconnection model (OSI model), the TCP/IP model, or any other suitable model.

“Comprises” means includes but is not limited to.

“Comprising” means including but not limited to.

“Computer” means any programmable machine capable of executing machine-readable instructions. A computer may include but is not limited to a general purpose computer, mainframe computer, microprocessor, computer server, digital signal processor, personal computer (PC), personal digital assistant (PDA), laptop computer, desktop computer, notebook computer, smartphone (such as Apple's iPhone™, Motorola's Atrix™ 4G, and Research In Motion's Blackberry™ devices, for example), tablet computer (such as Apple's iPad™ device, for example), netbook computer, portable computer, portable media player with network communication capabilities (such as Microsoft's Zune HD™ and Apple's iPod Touch™ devices, for example), camera with network communication capability, wearable computer, point of sale device, or a combination thereof. A computer may comprise one or more processors, which may comprise part of a single machine or multiple machines.

“Computer readable medium” means a tangible, non-transitory article of manufacture having a capacity for storing one or more computer programs, one or more pieces of data, or a combination thereof. A computer readable medium may include but is not limited to a computer memory, hard disk, memory stick, magnetic tape, floppy disk, optical disk (such as a CD or DVD), zip drive, or combination thereof.

“Having” means including but not limited to.

“Interface” means a portion of a computer processing system that serves as a point of interaction between or among two or more other components. An interface may be embodied in hardware, software, firmware, or a combination thereof.

“I/O device” may comprise any hardware that can be used to provide information to and/or receive information from a computer. Exemplary I/O devices may include disk drives, keyboards, video display screens, mouse pointers, joysticks, trackballs, printers, card readers, scanners (such as barcode, fingerprint, iris, QR code, and other types of scanners), RFID devices, tape drives, touch screens, cameras, movement sensors, network cards, storage devices, microphones, audio speakers, styli and transducers, and associated interfaces and drivers.

“Medical device” refers broadly to any apparatus, machine, or article of manufacture used in performing one or more actions on a patient, such as medical, dental, orthodontic, or similar procedures, for example. The patient may be a human or an animal.

“Medical device representative” or “representative” as used herein refers broadly to a person knowledgeable in at least one aspect of the operation or use of a medical device. A representative may, for example, be trained to help guide medical personnel in setup, operation, assembly, installation, accessing support data, or other operations with respect to a medical device. A representative may, for example, be a person trained to teach a new or existing application or protocol developed for a medical device. A representative may be a person specifically employed and/or trained by a medical device manufacturer or supplier or distributor. A representative may also be some other person trained in at least one aspect of the operation or use of a medical device who may not be specifically employed by a medical device manufacturer or supplier or distributor, such as an independent consultant, for example.

“Medical procedure” refers broadly to any method or process performed on a patient for healthcare purposes, such as a medical, dental, or orthodontic patient. For example, a medical procedure may be provided to treat, diagnose, or measure a patient condition. The patient may be a human or an animal. A medical procedure may, for example, be performed by a medical doctor, surgeon, radiologist, interventionalist, cardiologist, pain management physician, doctor of osteopathic medicine, physician assistant, nurse practitioner, or another medical professional.

“Memory” may comprise any computer readable medium in which information can be temporarily or permanently stored and retrieved. Examples of memory include various types of RAM and ROM, such as SRAM, DRAM, Z-RAM, flash, optical disks, magnetic tape, punch cards, EEPROM, and combinations thereof. Memory may be virtualized, and may be provided in or across one or more devices and/or geographic locations, such as RAID technology, for example.

“Network” refers to any type of communication medium and may comprise a cellular network, the Internet, intranet, local area network (LAN), wide area network (WAN), Metropolitan Area Network (MAN), other types of area networks, cable television network, satellite network, telephone network, public networks, private networks, wired or wireless networks, virtual, switched, routed, fully connected, and any combination and subnetwork thereof. A network may use a variety of network devices, such as routers, bridges, switches, hubs, repeaters, converters, receivers, proxies, firewalls, translators and the like. Network connections may be wired or wireless, and may use multiplexers, network interface cards, modems, ISDN terminal adapters, line drivers, and the like. A network may comprise any suitable topology, such as point-to-point, bus, star, tree, mesh, ring, and any combination or hybrid thereof.

“Operating room” means any facility in which a medical procedure is performed. In some embodiments, an operating room may comprise an interventional suite such as may include equipment for medical imaging.

“Patient Sticker” means any patient identifier such as a patient reference sticker, bracelet, or other identifier that may contain personal health information for the purposes of tracking, billing, or invoicing by vendors, physicians, hospitals, or other medical entities.

“Program” may comprise any sequence of instructions, such as an algorithm, for example, whether in a form that can be executed by a computer (object code), in a form that can be read by humans (source code), or otherwise. A program may comprise or call one or more data structures and variables. A program may be embodied in hardware, software, firmware, or a combination thereof. A program may be created using any suitable programming language, such as C, C++, Java, Perl, PHP, Ruby, SQL, other languages, and combinations thereof. Computer software may comprise one or more programs and related data. Examples of computer software may include system software (such as operating system software, device drivers and utilities), middleware (such as web servers, data access software and enterprise messaging software), application software (such as databases, video games and media players), firmware (such as software installed on calculators, keyboards and mobile phones), and programming tools (such as debuggers, compilers and text editors).

“Real-time” means a substantially simultaneous timeframe in which a computer system's activities substantially match the human perception of time.

“Signal” means a detectable physical phenomenon that is capable of conveying information. A signal may include but is not limited to an electrical signal, an electromagnetic signal, an optical signal, an acoustic signal, or a combination thereof.

Systems and methods are described herein for supporting medical procedures by allowing medical device representatives access to real-time information concerning an ongoing medical procedure (e.g., a surgical procedure) or demonstration of a medical procedure and providing for audio and visual communications between the medical device representatives and medical personnel involved in the medical procedure (or participating in a demonstration of the medical procedure) concerning how to use the relevant medical devices. Such systems and methods may, for example, provide live streaming and interactive capabilities for audio and visual communication between medical personnel and a representative. In some embodiments, a plurality of communication links between different locations may be established. The plurality of communication links may, for example, coordinate one or more surgical sites with one or more representatives, whether present in the proximity of a patient being operated on (e.g., in a hospital but outside of an operating room or within an operating room but not in close proximity to a patient) or at some other location. Audio and visual data described herein may be generated, captured, stored, transmitted, received, played, or displayed either separately or combined. Visual data may include still images, video images, or a combination thereof.

The systems described herein may include programmed or programmable computer devices including one or more computer programs embodied in one or more computer readable mediums including instructions for execution of the various functions and methods described herein. The programs and/or program parts described in connection with the embodiments disclosed herein may be implemented in electronic hardware, software, firmware, or a combination thereof.

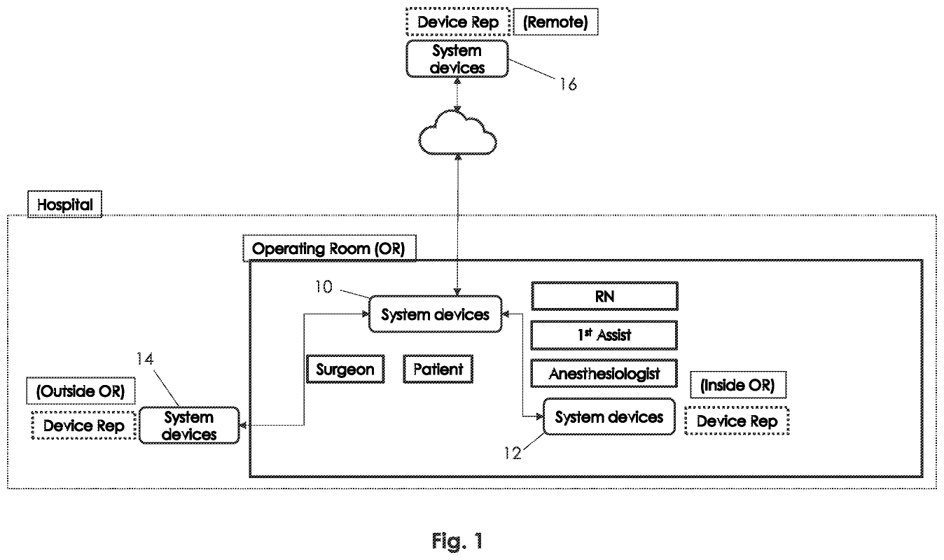

FIG. 1 is a schematic diagram showing a hospital or surgical center and one or more different devices or groups of devices (10, 12, 14, 16) (e.g., one or more computer devices having a video camera, display, microphone, and speaker) which may be provided to different persons to support a surgical procedure. Although a surgical procedure is described in this example, it should be understood that the procedure could be any medical procedure. As shown in FIG. 1, a number of communication links may be established between different persons and locations. A surgical center or hospital may, for example, be provided with a group of devices 10 configured for use in an operating room. The devices 10 may, for example, include one or more communication devices configured for collecting visual and/or audio data generated within an operating room, sending collected visual and/or audio data to one or more representative devices, or both. The communication devices 10 may further be configured for receiving instructions and/or other data (e.g., audio and/or video data) from one or more representative devices 12, 14, 16. In some embodiments, the group of devices 10 may further include, by way of nonlimiting example, one or more mounting devices (e.g., adjustable holders or robotic arms for positioning communication devices), cameras, monitors, computer processors, computer styluses or pens, and other devices suitable for executing the methods described herein. Control of the various system operations and related method steps described herein may be executed using one or more computer processors. Computer processors may be physically housed together with other device components (e.g., an iPad™ communication device may include a computer processor and also include a video camera) or a computer processor may be housed separately while in operable communication with other device(s) described herein.

A communication device may, for example, comprise an iPad™ tablet computer, iPhone™ smartphone, iTouch™ wearable smartwatch, or other computer device that may include or be operably connected to a video camera. A communication device may include an integrated computer processor suitably configured to control the video camera or to perform other system functions. A video camera may also receive one or more control signals from another device. Communication devices may interface with one or more routers so as to be connected to the internet via a surgical center WIFI, ethernet, or cellular network, for example. To provide for redundancy, a group of communication devices may be connected to different cellular networks, for example.

A video camera may be positioned on an adjustable mount. For example, the position of a video camera may be adjusted by securing an iPad™ device in a mount (e.g., such as may include one or more clips, suction elements, magnets, or slotted features for holding the iPad™ device) that enables the video camera to be held in a desired position. The adjustable mount may further be configured so that the video camera may be moved or positioned through one or more degrees of freedom, such as horizontal and/or vertical tilting, for example, or any combination of the available six degrees of freedom (three translations and three rotations) in a Cartesian coordinate system. Of course, any suitable reference frame may be used for the translational position and angular orientation of the video camera or device that includes the video camera. In some embodiments, a video camera may be positioned using an adjustable mount including or coupled to a robotic arm. A robotic arm may, for example, be configured to execute one or more of a pivoting, rotating, or translating adjustment so as to allow a user to control and position a video camera to a preferred angle or position to view a medical procedure. A robotic arm may, for example, include one or more actuators, servomotors, or other devices suitable to control the position of a camera by controlling pivoting, rotating, or translating adjustment of a video camera. An adjustable mount or robotic arm may be interfaced with one or more of the communication devices 10 described herein. For example, an iPad™ computer may be positioned within an operating room and its video camera focused on one or more persons or structures therein. An adjustable mount or robotic arm on which the iPad™ computer is mounted may receive an input or control signal from the iPad™ computer so as to actively maintain or optimize focus or positioning of the video camera on the one or more persons or structures. Alternatively or additionally, an adjustable mount or robotic arm (or a communication device 10 in communication therewith) may receive a control signal from a representative device 12, 14, 16 to control a position and/or orientation of the video camera, its field of view, its focus, or other operating characteristic of the video camera, or a combination thereof. For example, in some embodiments, an adjustable mount or robotic arm may be in communication with a representative device so that the representative may take active control of the camera of the iPad™ device in the operating room to remotely zoom in on a critical aspect of the surgery or to better position the camera when taking a photograph of a region of interest for editing or mark-up so as to communicate one or more aspects of a medical device or procedure to personnel in the operating room.

In some embodiments, an adjustable mount or robotic arm may be configured to automatically follow one or more functional aspects of a medical procedure in an intelligent manner. A functional aspect of a medical procedure may, for example, refer to a position of a person or thing (e.g., medical device or piece of equipment) involved in the medical procedure or the orientation or relative position of two or more things involved in the medical procedure. In some embodiments, a functional aspect of a medical procedure may include a position of a surgeon, a position of a surgeon's hand, a position of an exposed portion of a patient's body, a position of an incision on a patient's body, a position of a medical device, or a position of a medical device diagnostic screen. For example, a system may be configured to use facial recognition or object detection to identify the position of a surgeon so that a camera may be automatically adjusted to ensure that the surgeon is in view. Accordingly, an image of the surgeon may be continuously transmitted to a supporting medical device representative. In some embodiments, a system herein may also be configured to follow other aspects of a medical procedure, including, for example, surgical incision sites and medical devices or parts thereof, such as data screens. Selection of functional aspects of a medical procedure may, for example, be made at the start of a medical procedure or part of a medical procedure. Accordingly, areas or persons of interest associated with a medical procedure may be selected and automatically tracked by one or more cameras during a procedure. In some embodiments, a number of functional aspects of a medical procedure may be tracked, such as may involve use of more than one video camera. Systems herein may further be configured to indicate the hospital location, floor, and room wherein a video camera or other device is located.

Systems herein may include one or more video display monitors (sometimes referred to herein as a screen). Monitors may, for example, be included in one or more of the iPad™ devices described above as also used as video cameras. A dual iPad′ configuration may, for example, provide a first screen to be dedicated to providing a live video stream of the supporting representative so that the surgeon knows at all times the representative is effectively “there in the room” with them as well as a picture-in-picture (PIP) or other display of the camera view that the representative is seeing so that the surgeon can make sure the representative can see the case properly. One or more additional iPad™ devices or monitors may also be used for executing other features of the system including, for example, invitation of other representatives, display of information provided from the representative such as marked up photographs or other information relevant to the procedure or for executing other system features. An additional monitor may, for example, be provided as an iPad™ device suitably programmed to receive instructional information such as “visual aids” from a representative. Such instructional information may, for example, include screen shots provided from one or more cameras (e.g., screen shots selected and, in some cases, marked up or annotated by a representative), instructional videos, textual messages, or other information. For example, a screen captured from an iPad™ camera may be displayed on a monitor as marked up by the representative (e.g., using their finger or “markup drawing tools”) such as to include lines, circles, or other features to point to and highlight things on which they desire to instruct the surgeon. In some embodiments, the screen may be used to project an avatar representation of a tool on a captured image, for example. An avatar representation of a tool may, for example, be positioned on an image manually by an operator. Alternatively, placement of an avatar representation of a tool may be accomplished using computer vision technologies, such as may include execution of one or more protocols to automatically perform image analysis (e.g., computer guided object analysis) to facilitate automatic placement or positioning of a virtual object on an image. For example, computer vision technologies may be used to identify an anatomical feature on an image (e.g., the position of a hip joint) and an avatar representation of hip implant may be automatically positioned at a desired position or orientation with respect to the imaged hip joint.

In some embodiments, a video camera or separate optical device may be equipped to measure distances to and between objects or object points within its field of view. For example, the video camera or other device may be equipped with a pulsed light source and detection optics suitable for measuring a time of detection of scattered light to establish distance to an imaged point on an object. For example, an iPad™ camera may be a LIDAR camera suitable to perform distance measurements and thus allowing the representative to remotely take advantage of the LIDAR camera on the iPad™ device in the operating room to perform measurements and show them to the physician. Avatar devices or other marked up features may further be proportioned against one or more measured objects or distances in a visual field so as to virtually project a device in correct proportion with respect to objects in a camera's field of view. In some embodiments, a video camera or other optical device may be designed to operate at some distance or within a specified range so as to properly assign measurement distances to image features collected therein and to scale marked-up or avatar features added thereto. And, in some embodiments, systems and methods herein may be designed to warn a user when collected visual data may be outside of a recommended range or within a desired or acceptable range, adjust a focal distance or angle to minimize errors in a measured dimension, or both.

In some embodiments, one or more communication devices 10 and representative devices 12, 14, 16 may include spatial awareness capabilities, such as the nearby interaction functionality in the U1 chip and MySpatialApp™ available from Apple Inc. (Cupertino, Calif.). In such embodiments, each of the communication devices 10 and representative devices 12, 14, 16 may determine distance and direction information of that device relative to any of the other devices, or vice versa. That relative distance and direction information may be used to provide various useful functionalities. For example, relative distance and direction information may be used to add a level of security when connecting medical device representatives to a case because representative devices 12, 14, 16 may have to be in physical proximity to the communication devices 10 in order to join the case. This information may further be logged and used to verify that a medical device representative actually participated in a procedure. This information may be included or required when generating or receiving purchase orders, invoices, or other related documents and files. Relative distance and/or direction information may be electronically logged and used to help identify fraudulent or incorrectly generated or received purchase orders, invoices, or other related documents and files. In some embodiments, the spatial awareness capabilities of one device with respect to another device may be operational only if the other device grants permission to the one device. In some embodiments, such permission may be granted via an electronic token that is valid only during a current session of the application, and once the application is closed or the current session is otherwise ended, the spatial awareness with respect to the device that granted such permission will be ended. In some embodiments, such spatial awareness may be provided only if the participating devices are within a certain distance from one another and there is an unobstructed line of sight between the participating devices. In other embodiments, such spatial awareness may be provided between or among participating devices that are remote from one another and/or not within an unobstructed line of sight.

In some embodiments, one or more monitors may be used in a “whiteboard” mode wherein a representative may project information to the monitor (or iPad™) as if drawing on the board in free hand just like if there was a white board in the operating room. For example, a representative could write down steps in a protocol for medical personnel to execute. Medical personnel may view the information in real time as it is added. In some embodiments, medical personnel may further be able to use the whiteboard. For example, medical personnel may be able to ask questions using the whiteboard or to point to or circle steps in a protocol wherein they may have questions or concerns. Systems herein may further record which party (e.g., medical device representative or medical personnel) has added a note or marking.

In some embodiments, systems herein may have access to an asset library such as may include representations of medical equipment (e.g., 3D representations of medical device products that may be projected on a second iPad™ monitor) so as to provide an augmented reality depiction of the medical equipment. For example, a representative may be remotely looking through the camera and able to see a virtual object representing their product or a tool or implement that may be used together with their product. In some embodiments, the representative may be remotely looking through the camera and able to see a virtual object representing their product and move or project the object through a screen shot or captured video collected during a surgery, which may also be viewed by medical personnel in the operating room via the one or more communication devices 10. For example, using a representative device 12, 14, 16, a representative may mark one or more anatomical reference points from a captured image received from a communication device 10 and initiate movement or positioning of a virtual depiction of a medical device so as to orient the virtual depiction of the medical device in relationship to the one or more anatomical reference points. The captured images together with the virtual depiction of the medical device may likewise be received by one or more communication devices 10 of medical personnel (e.g., within an operating room) and displayed for view therein. In some embodiments, the 3D objects may also be used as virtual indicators, measurements, call outs, or the like that exist in a 3D virtual world so even if the position or orientation of the iPad™ device is changed, these indicators would hold their position. In some embodiments, the system may save a record of the items used in the procedure, such that the representative may submit an invoice to the hospital or other responsible party for items that were used in a given surgical case.

Systems herein may further include devices or a group of devices (12, 14, 16) configured for use by one or more representatives. For example, a representative may be a technician available on-site to assist medical personnel, such as a surgeon or other surgical staff. Surgical staff may, for example, include one or more attending surgeons, registered nurses, assistant personnel, and other doctors such as anesthesia doctors. In some embodiments, it may be useful to have the representative available within an operating room (as shown at 12) or outside of an operating room (as shown at 14), such as in an adjoining room or other location in a hospital. In both cases, the devices 12, 14 may enable the representative to interact with surgical staff via audio and video communications while minimizing risk of inadvertent spread of microbes. In some embodiments, a group of devices 16 may also be provided to one or more remotely located representatives. The group of devices 12, 14, 16 may include one or more computers configured for receiving audio and visual data regarding a surgery from communication devices 10 and for providing instructions for use of a medical device to surgical staff using communication devices 10 in real-time during the procedure in which the medical device is being used. For example, a group of devices 12, 14, 16 may include a computer with an I/O device such as a computer pen or stylus. A stylus or other suitable I/O device may allow a representative to provide mark ups or provide notes on a document or image so that information may be provided to surgical staff for viewing on the communication devices 10 in the operating room.

Methods for supporting surgical procedures are further described herein. In execution of these methods, hospital administration may, for example, create an account and set up billing using a software-as-a-service (SAAS) protocol. Computer programs and applications for executing the methods described herein may then be accessed and used over a network, for example. In some embodiments, one or more computer applications for executing methods described herein may be installed to run on a computer (e.g., devices 10, 12, 14, 16) in guided access mode (“kiosk mode”). In this mode, the application may run continuously without being closed.

In some embodiments, a surgeon or care team may set up or login to an existing account using a communication device 10, such as an iPad™ computer, for example. In some embodiments, a surgeon or care team member may launch an application on their own device or a pre-registered iPhone™ or iPad™ device equipped with Face ID™, for example. A login may be executed before the medical staff member has placed a surgical mask on his or her face or by temporarily removing a facial mask if needed. A medical team user may further invite one or more representatives to support a given medical procedure. Systems and methods herein may enable a user to set up or access a group of pre-authorized representatives that they may want to invite to assist in a surgery or other medical procedure. In some embodiments, when surgery begins, an application user (e.g., nurse, surgeon, or another care team member) may start a new case and confirm they are in an identified operating room based on its settings. To assist in identification of a room setting, a communication device 10, 12, 14, 16 may be equipped with GPS or other suitable means of room identification may be used, such as selection of a room from a menu, or entry of room data in a text field, for example.

The user may then be provided any of several ways for sharing the case in progress and inviting one or more device representatives to participate. In some embodiments, a communication device 10, 12, 14, 16 may be programmed to automatically identify nearby communication devices associated with representatives that are in physical proximity and display associated usernames for the associated representatives. The communication device may further send a case request to a representative so that the representative may accept or otherwise communicate that they are or are not available. If no representatives are detected as being in physical proximity, an invitation code may be texted or emailed to one or more representatives. In some embodiments, methods herein may provide access to a registry of qualified representatives to provide an on-demand consulting service of available and qualified representatives. Some methods herein may further allow multiple representatives to be added in a “group mode” setting.

In some embodiments, a surgeon (or other operator of a communication device) may position the device 10 in between the surgeon and a patient or medical device, such that the surgeon may be looking at the case (e.g., patient and/or medical device) through the associated camera of device 10, which may provide an opportunity for the representative to use an augmented reality (AR) mode of operation on device 12, 14, 16. In this mode of operation, the representative may use symbols, display measurement tools, or project an instrument or medical device on communication device 10 from representative device 12, 14, 16 using an augmented reality mode. For example, a user may view virtual features displayed upon the real environment using communication device 10 which may comprise augmented reality glasses or some other viewing device. A specific example of such augmented reality glasses is Google Glass (a trademark of Google, Inc.). Augmented reality glasses may further include those made available from Apple Inc., for example. In this way, a representative may, for example, point to something on the patient and/or medical device without risking infection of the surgery site. This “virtual pointing” may be used to communicate instructions more easily and accurately to the surgeon or other member of the care team. To assist in virtual projection or pointing, a representative device 12, 14, 16 may have a number of symbols and medical devices pre-programmed for quick and easy display. For example, a representative device may have a pre-programmed kit of avatar images of various instruments and tools such as implants, sutures, or other medical instruments. The avatar images may, for example, be statically displayed or may be dynamically displayed through one or more user-defined or pre-programmed motions or actions in this AR mode. Other data may be displayed in an augmented reality environment. A virtual reality system or augmented reality system may, for example, receive input data in various forms including in the form of visual, sound, voice commands, LIDAR image data, or from one or more medical devices, machines, or databases. Visual data, sound data, voice data, augmented reality data, and virtual reality data may be captured, for example, using a sound transducer or microphone, computer configured to execute computer vision technology, LIDAR imaging system, or other suitable data capture system. In some embodiments, medical device data may be received from patient monitors, X-ray machines, magnetic resonance imaging (MRI) equipment, computerized tomography equipment, picture archiving and communication systems (PACS), internet of things (IOT) sensors, medical device sensors. A processer of the virtual reality system may receive these inputs and process the data so as to command an augmented reality display device (e.g., augmented reality glasses) to project the data for viewing.

In some embodiments, the systems and methods herein may be used for providing post-operational instructions where there is a concern with unnecessarily exposing a patient to infectious microbes. The system and methods herein may also be used to provide information to a patient at some other time. For example, instructions may be provided to a patient before surgery or during treatment of a disease where it may be useful for a surgeon or representative to be outside of a patient's room so as to keep the patient from being exposed to microbes. A doctor or other care member may, for example, be outside the door of a patient's room containing device 10 and communicating with the patient from devices 12, 14, 16.

In some embodiments, a first member of a medical care team may be at a central desk of a floor of a hospital and a second member of a team may carry a device 10 with them when entering a patient's room. A first care team member may, for example, be a more senior member and a second care team member may be a less experienced member who is administering a drug (e.g. chemotherapeutic drug) to a patient who may be immune compromised. In this way, the more senior staff member may consult with any of various team members as needed while those other team members provide care.

In some embodiments, the systems and methods herein may be used by a surgeon or other care member to obtain patient vitals. For example, any medical provider with proper authorization could temporarily log on to a communication device 10, 12, 14, 16, such as an iPad™ computer, and obtain medical data without requiring the provider to be in the room with the patient.

In some embodiments, a representative may use the systems and methods herein by creating a user account. One or more security protocols may be implemented to ensure only authorized individuals are signed up or otherwise invited to a surgery. In some embodiments, one level of security authorization may be implemented by requiring that a representative device 12, 14, 16 is within close proximity to a surgical center. Security protocols may sometimes be tailored for whether or not a device 12, 14, 16 is within proximity to a surgical center. In some embodiments, systems and methods herein may only display cases that are “active” (either started or being triaged), and when a representative asks to join, a staff user may be given an option to accept that representative.

In some embodiments, a representative may be required to send a picture or video of himself or herself. For instance, an application may require a representative to send a current picture of himself or herself. For example, the application may only allow a new camera image (not an image from a camera roll) to validate that a representative is who they say they are and not someone using someone else's login to gain unauthorized access. This may also provide a way when the vendor with which the representative is associated bills the hospital to confirm that the representative that they are billing for was indeed at the case. For example, the vendor may provide a timestamped photograph of the representative, geographical data, or both to confirm the representative was virtually present and participated in the surgery. Systems and methods herein may display relevant information for the representative to know they are connecting to the correct case without providing personal health information (PHI). However, log in and communication protocols may help protect PHI that may otherwise be inappropriately distributed.

In some embodiments, communication devices 10, 12, 14, 16 herein may be used to watch a large number of surgeries and may collect data and learn about the procedures they are watching. The systems may analyze various things, including personnel involved, medical devices used, how long different parts of the procedure are taking, and the like, which when correlated with outcomes, may help inform research on the likelihood of better outcomes. This information may also be integrated with pre-operative information and the ability to load up complications/successes for patients of the same demographic/procedure such as to identify any complications having happened for a certain patient type or medical device. In some embodiments, systems and methods herein may be used to perform predictive analytics resulting in improved cost of care and procedure efficiencies, and improved clinical outcomes for medical procedures.

In some embodiments, computer programs herein may use voice recognition to provide automatic closed captioning for the representative to assist them in understanding what is being said in the operating room, particularly by the surgeon.

In some embodiments, systems herein may use natural language processing (NLP) to create automatic/live searching of the equipment documentation and present it contextually on the iPad™ device 10, 12, 14, 16 and/or a connected monitor. In that way, as the doctor is proceeding through the surgery, the system may aid in providing context for the provider and the representative.

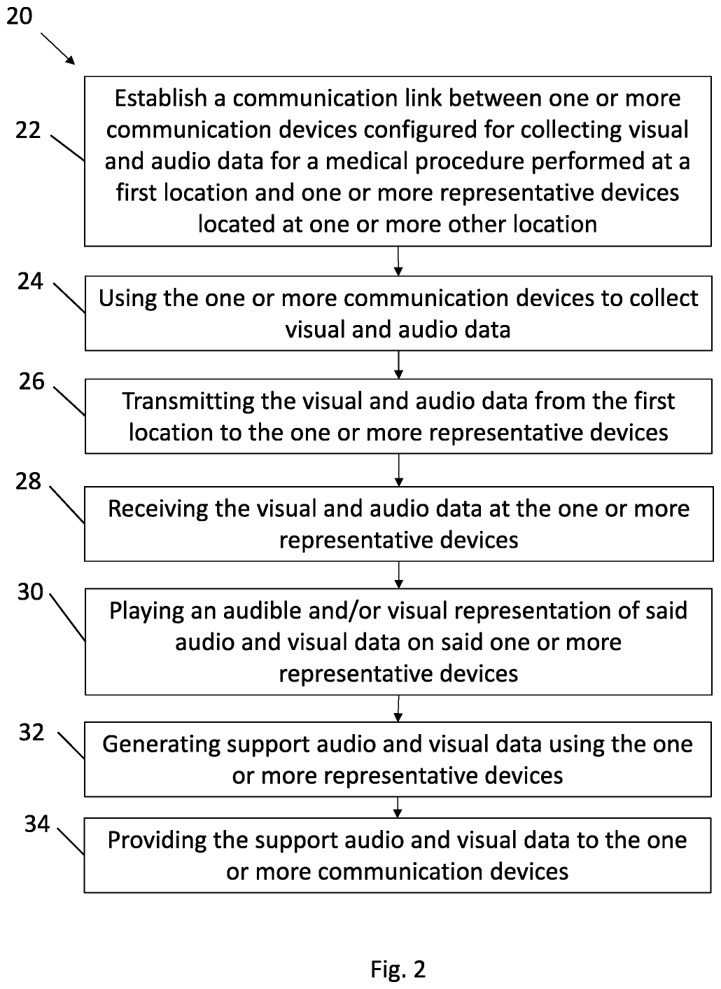

FIG. 2 shows an embodiment of a method 20 for supporting a medical procedure by providing a medical device representative access to real-time information concerning the medical procedure and for providing audio and visual communication to a doctor or other medical personnel such as a surgeon, radiologist, interventionalist, cardiologist, pain management physician, doctor of osteopathic medicine, physician assistant, nurse practitioner, or another medical professional concerning how to use the relevant medical devices. As shown in a step 22, one or more communication links may be established between one or more communication devices configured for collection of audio and visual data for a medical procedure at a first location and one or more representative devices located at one or more other locations. The first location may, for example, be an operating room or other hospital room housing a patient. In some embodiments, a first location may be an interventional suite, specials suite, procedure room, office based lab/procedure room, or another space where a medical procedure takes place. A patient may, for example, be a person prepared for surgery, a person with a weakened immune system, a person who may have a contagious condition, or another person where it may be advantageous to minimize physical proximity between the person and others.

In some embodiments, a communication link may be established by identifying nearby representative devices that are in physical proximity to the first location. Physical proximity in the context may refer to the condition wherein a communication device may detect a representative device, such as a communication device and representative device are both connected to a common local area network. For example, a communication device 10 may automatically identify nearby representative devices associated with representatives that are in physical proximity to the first location and display associated usernames. One or more requests to join the case may then be sent. If no nearby devices are identified and/or if no representatives accept a request to join a case, an invitation code may be sent (e.g., by text or email) to a more expanded group of representatives (e.g., a group of representatives who may not be in physical proximity to the first location). In some embodiments, a group of preferred representatives may be identified and provided a first option to join a case. For example, one or more preferred representatives may be provided with a first option to accept a case invitation before other representatives are invited. In some embodiments, an active registry of qualified representatives or qualified representatives who also pay for registry may be accessed when a new case is created. And, in some embodiments, a plurality of medical device representatives may be added to interface with a medical procedure in a group mode.

As shown in the step 24, one or more communication devices may be used to collect audio and visual data. For example, one or more functional aspects of interest for the medical procedure may be identified and one or more video cameras and/or microphones may be positioned appropriately to monitor the one or more functional aspects. In some embodiments, a first functional aspect of a medical procedure may comprise the position of an attendant surgeon. An attendant surgeon may, for example, be identified as an object to be tracked during a surgical procedure. A first video camera or microphone may be focused on the surgeon so that the representative may be able to see and/or hear the surgeon and respond to any verbal or other requests for information or instructions from the surgeon. A second video camera may be focused on one or more other functional aspects of a medical procedure, including, for example, an exposed portion of a patient's body or incision site, a medical device, a diagnostic data screen, or any combinations thereof. Selection of functional aspects of a medical procedure may be made at the start of a medical procedure or made or changed during the course of a medical procedure as may be needed. For example, if one attendant surgeon is leading a case during a first part of a medical procedure but another attendant surgeon is leading the case for a second part of a medical procedure, video cameras may be adjusted accordingly. A selected group of one or more functional aspects of a medical procedure may then be tracked or followed. For example, if a video camera is tasked with tracking a selected object and the object moves or is moved, a communications device may detect that the object is no longer in view and automatically adjust a focus, tilt, or camera position to ensure the functional aspect remains in view. In another example, a first video camera may be focused on an attending surgeon. A second video camera may be under a medical device representative's control so that the representative may actively position the camera as necessary to capture relevant audio and/or visual data of subject matter wherein explanation may be useful.

As shown in a step 26, the collected audio and visual data may be transmitted from the first location so that it may be received at one or more other locations. Visual data may, for example, comprise computer vision data, light or sound emitted data, LIDAR data, Video data, augmented reality data, virtual reality data, holographic images, other data as described herein, and any combinations thereof. More generally, data may be provided between any number of different sites. For example, for some complicated medical procedures, data may be provided between or among a plurality of operating rooms or supporting locations in one or more hospitals and a plurality of remote locations. By providing data between a plurality of operating rooms or supporting locations in one or more hospitals and other remote locations peer-to-peer collaboration or collaboration between different groups (e.g., peers, patients, family, or other industry members) may be facilitated. Peer-to-peer collaboration may refer to a collaboration between any number of physicians in proximity or a virtual meeting.

Data transmitted from a first location may generally include audio and visual data collected from one or more communication devices such as may include a microphone and a video camera. However, other data may also be sent from a first location. For example, in some cases, a medical device itself may send additional data to a remote medical device representative through a separate wired or wireless linkage. However, it should be understood that a key advantage of some methods and systems herein is that information may be captured from a medical device screen and sent from a communication device 10 to a representative device 12, 14, 16 without having to establish a separate communication link between the medical device and a remotely located medical device representative. Accordingly, data from various different medical devices may be readily communicated from communication device 10 using a common interface without having to worry about establishing separate communication links for different medical devices.

As shown in a step 28, collected visual and/or audio data, or at least an appropriate subset of the collected visual and/or audio data (e.g., a subset of the collected data suitable for sharing with one or more participating representatives), may be received at the one or more other locations using one or more of the representative devices or group of devices (12, 14, 16). Thus, visual and/or audio data may be provided or selectively provided between the first location and the one or more other locations. In some embodiments, one or more of the devices (12, 14, 16) may be configured to process received data, such as to convert the data to a readable form or to help mine the data for useful data features. For example, visual and/or audio data from a medical device may be processed using natural language processing (NLP), or optical character recognition (OCR) may be applied so that alphanumeric information may be digitized. As an alternative to NLP or OCR processing of received data, this processing may also be performed prior to data transmission using one or more of the devices or group of devices 10. Thus, NLP or OCR processing may be performed by one or more computer processors included among any of the devices or group of devices (10, 12, 14, 16). For example, video cameras herein may focus on one or more display screens of a medical device. The display screen may present alphanumeric or graphical data. This data may be subject to OCR or other processing, such as may, for example, facilitate qualitative or quantitative comparison of the data to one or more control limits or conditions. A medical device representative may then track this information during a medical procedure. For example, one or more of the devices 12, 14, 16 may execute a sub-method to perform OCR on received alphanumeric data from a remotely positioned medical device and to compare the data to one or more control limits or condition values so as to identify a relevant condition. As appropriate, a medical device representative may interrupt a surgeon so as to warn him or her of the condition, for example.

As shown in step 30, an audible and/or visual representation of the audio and visual data from communication device 10 may be played or displayed by the representative device 12, 14, 16 for the associated representative supporting the medical procedure. In response to the played or displayed audible and/or visual representation of the audio and visual data, the medical device representative may generate (e.g., by creation, selection, or otherwise) support audio and visual data as shown at step 32. For example, the medical device representative may play or display the audible and/or visual representation of the audio and visual data so that they are aware of the stage or part of the medical procedure where a medical device may be used or used in a particular way. Further, by playing or displaying an audible and/or visual representation of the audio and visual data, the medical device representative may be aware of any questions presented by medical personnel and respond to the questions in real time.

In some embodiments, capability for manual editing of received visual and/or audio data may be provided at representative device 12, 14, 16. For example, a medical device representative may receive the audio and visual data, play an audible and/or visual representation of the audio and visual data, and isolate one or more relevant images, videos, or audio streams from the audio and visual data. A representative device 12, 14, 16 may further be programmed to select one or more portions of the audible and/or visual representation so as to isolate particular portions included therein and create an image file, a video file, an audio file, or an audiovisual file from the data. The representative may be provided access to an asset library such as may include representations of surgical equipment (e.g., 3D representations of medical device products that could then be projected on a second iPad™ monitor) and the representative may be given an option to select one or more of the assets. The representative device may support insertion of the asset into an isolated image or video file so as to provide a static or dynamic display of the asset within the context of the medical procedure. Accordingly, a medical device representative may create a virtual depiction of an asset within the audio and visual data collected during the medical procedure. This virtual depiction of an asset within the audio and visual data may be referred to as a virtual data file. In another example, a representative device may be configured to mark up one or more created image files and/or video files. For example, the audio and visual data may be marked up by the representative (e.g., using their finger or “markup drawing tools”) such as to include lines, circles, or other features to point to and highlight things on which they desire to instruct medical personnel. In another example, a representative device may be configured to provide one or more audio files such as may be used to communicate to medical personnel a sound they should expect to hear when using a device. Accordingly, edited or marked up audio and visual data may be created. Other instructional material may further be created or selected. For example, a “whiteboard” may be created so that a representative may draw on the board in free hand. For example, a representative could write down steps in a protocol for medical personnel to execute. A whiteboard may further be used to give context or answer any questions about other instructional materials which may be shared with medical personnel. Or, one or more previously created data files, including, for example, a predefined audio file, image file, video file, or audiovisual file may be selected.

In a step 34, support audio and visual data may be provided to the one or more communication devices 10 from one or more representative devices 12, 14, 16. For example, any combination of edited or marked up audio and visual data, one of more virtual data files, and other instructional materials may be provided from the one or more representative devices 12, 14, 16 to the one or more communication devices 10. In some embodiments, one or more of previously created data files may be stored on the one or more communication devices so that only a link or instruction to the data file may be provided by a medical device representative. This protocol for providing the material may be particularly useful for large files or in situations where communication bandwidth may be limited.

In some embodiments, systems and methods herein may record a video of the surgery which can be used to confirm and prove the actual devices used in the procedure to help prevent errors and/or fraud. As the surgeon is using various devices, the representative can build an invoice listing the relevant devices used in real-time. Device inventories can be updated as devices are used. In some embodiments, use of a device may be logged by scanning a device barcode or other device identifier, logged when a communication device camera recognizes use of a device, or both. A camera may, for example, be used to take a picture of an identification number of a device. Optical character recognition may then be used to record the identification number. The recorded identification number may then be used to log that the device has been used. Alternatively, automatic identification of a device and logging of its use in a medical procedure may be based on video recording of the procedure and identification of the device based on recognition of a property of the device, such as, for example, its shape. Device use may be time-stamped and associated with a unique case identification number. Thus, systems and methods herein may automatically generate data used for inventory tracking, thereby eliminating or reducing a need for manually updating an inventory. When a case is done, a nurse from the hospital may use systems herein to digitally sign off and approve an invoice for the items used. In some embodiments, systems and methods described herein may automatically create a purchase order or other document authorizing the use of the relevant devices for a particular medical procedure. In some embodiments, systems and methods herein may facilitate a workflow including automatic generation of a unique case identification code. For example, a unique case identification code may be created and distributed to a medical device representative. This identification code may be associated with a purchase order and used as a substitute for a patient sticker in medical billing. Therefore, systems and methods herein may be used to eliminate or minimize distribution of personal health information. An identification code can be cross referenced with other data collected during a medical procedure, including, for example, spatial identification data establishing that a representative device was within proximity to a certain procedure (e.g., near an operating room). Therefore, the systems and methods herein may also be used to reduce risk of fraud and reduce other errors in billing.

In some embodiments, systems and methods herein may collect data suitable for documenting a medical procedure or part of a medical procedure. Data collected for documenting a medical procedure may comprise visual data (e.g., video image data, LIDAR image data, or both), audio data, or a combination of both audio and visual data. Original or raw data collected during a procedure may be stored to document a medical procedure. Alternatively, or in addition to storage of collected original or raw data, collected data may be processed to determine data values associated with one or more aspects of a medical procedure. The determined values may then be stored. In some embodiments, stored visual data may be LIDAR data. Other visual data, if collected together with LIDAR data, such as color coded video image data may be stored together with the LIDAR data or discarded without being archived or stored. Audio data may include, for example, an audio recording provided by medical personnel (e.g., a recording of a surgeon announcing start or completion of a part of a procedure), a recording of sound provided by a medical device, a recording of sound provided by the patient (e.g., produced verbally or otherwise), or any combinations thereof.

Documenting a medical procedure may include, for example, determining one or more start times or end times for a procedure or part of a procedure. For example, a start time for a surgical procedure may be recorded when a patient is moved into an operating room. Other times recorded during a medical procedure may include, by way of nonlimiting example, a time in which an incision is made, a time in which a portion of bone or other internal tissues is removed or otherwise manipulated, a time in which an implant is first disposed within a patient's body, a time in which a contrast or other imaging agent is given to a patient, a time in which a suture or related wound closure device is used, a time in which an incision site is closed, a time in which one or more supporting radiologic or other images of an anatomical feature is obtained or otherwise provided to medical personnel, a time in which the patient is moved from an operating room, and any combinations thereof. It may be useful to document other times for different types of medical procedures. Other information may further be collected and used to document a medical procedure. For example, one or more diagnostic values (e.g., patient vital signs) may be recorded at any of the above times or at other suitable times in a medical procedure.

In some embodiments, visual data (e.g., video image data, LIDAR image data, or both) may be collected and processed to determine one or more measurements for an incision or related surgical opening, such as, for example, a length, width, depth, or angle. For example, an incision may be made by a surgeon to expose a portion of bone or other internal anatomical structure. One or more portions of the exposed bone may then be removed or separated, such as to create an opening wherein an implant may be inserted. The dimensions of such an opening (e.g., length, width, angle, or depth) and/or relative geometries of the opening to or between one or more other anatomical structures (e.g., the distance or angle to or between nearby joints or bones) may be determined. For example, an operator may visually define endpoints for an opening or other anatomical feature. In some embodiments, computer vision detection technology may be used to automatically define endpoints of a geometric distance or placement. The determined features may then be stored or documented. Stored information may, for example, be compared to outcomes and used to identify particular aspects of a procedure related to positive or negative outcomes.

In some embodiments, systems and methods described herein may provide a number of advantages. For example, representatives can support surgery without having to be physically present in an operating room. Representatives can utilize an extra iPad™ device to provide training, visual aids, diagrams, and assist a surgeon even better than if they were just in the room in a traditional way. Representatives can be in the vicinity of an iPad™ device via Bluetooth and/or local WIFI, for example. Notably, in some embodiments, systems and methods herein may not require internet service. However, if a representative device has internet or other network connectivity, a representative may be remote and still support a medical procedure. Junior representative/trainees may bring in their senior representative to assist in training remotely. In some cases, a specialty consult can be requested. The systems and methods herein may be secure/HIPAA compliant. Protection of personal information may be enforced (replacing need for patient stickers). Systems and methods herein may help ensure a representative/vendor gets paid sooner and reduce fraud by ensuring representatives were actually participating in a case. Systems and methods herein may further facilitate tracking and signoff of equipment used in the hospital. Systems and methods herein may further open up opportunities for surgical consults in real-time. Systems and methods herein may further be inexpensive for the hospital, easy to install/support, and decrease exposure to COVID-19 and other diseases.

The systems and methods are flexible and easy to use but also provide a very secure system to ensure only the properly authorized users are able to access and watch and communicate with the surgeon and care team for only the cases in which they are authorized to participate.

Although the foregoing specific details describe certain embodiments of this invention, persons of ordinary skill in the art will recognize that various changes may be made in the details of this invention without departing from the spirit and scope of the invention as defined in the appended claims and other claims that may be drawn to this invention and considering the doctrine of equivalents. Among other things, any feature described for one embodiment may be used in any other embodiment, and any feature described herein may be used independently or in combination with other features. Also, unless the context indicates otherwise, it should be understood that when a component is described herein as being mounted or connected to another component, such mounting or connection may be direct with no intermediate components or indirect with one or more intermediate components. Therefore, it should be understood that this invention is not to be limited to the specific details shown and described herein.

1. A system for supporting a medical procedure involving a medical device, the system comprising:
one or more communication devices including a microphone, a speaker, a video camera, and a display, said one or more communication devices configured to collect audio and visual data of an ongoing medical procedure within an operating room and to provide said audio and visual data to a representative device;
the representative device being configured to receive said audio and visual data, play an audible representation of said audio data, and display a visual representation of said visual data;
the one or more communication devices being further configured to receive support audio and visual data regarding the use of the medical device from the representative device, play an audible representation of said support audio data, and display a visual representation of said support visual data in real-time during the medical procedure.

2. The system of claim 1 wherein said one or more communication devices comprise an iPad™ device, said video camera being an integrated component included in the iPad™ device.

3. The system of claim 1 further comprising an adjustable mount, the adjustable mount configured for positioning said video camera through one or more degrees of freedom.

4. The system of claim 3 wherein said adjustable mount includes a robotic arm.

5. The system of claim 3 wherein said adjustable mount is configured for removable coupling to a robotic arm.

6. The system of claim 3 wherein said adjustable mount is configured to automatically position said video camera to maintain a view of a functional aspect of said medical procedure.

7. The system of claim 3 wherein said adjustable mount is configured to automatically position said video camera to maintain a view of at least one of a person, a medical device, and a medical device data screen.

8. The system of claim 6 wherein said one or more communication devices includes at least one processor configured to analyze said audio and visual data in order to provide a control signal to automatically position said video camera in order to monitor said functional aspect of said medical procedure.

9. The system of claim 6 wherein said functional aspect of said medical procedure comprises at least one of a position of a surgeon, a position of a surgeon's hand, a position of an exposed portion of a patient's body, a position of an incision on a patient's body, a position of a medical device, and a position of a medical device diagnostic screen.

10. The system of claim 1 further comprising a robotic arm configured for positioning said video camera, the robotic arm configured to receive an input so as to actively maintain a focus of said video camera on a person or medical device involved in said medical procedure.

11. The system of claim 1 wherein said video camera is configured to receive a control signal from either or both of said representative device or another remote device under a representative's control.

12. The system of claim 1 wherein said video camera is configured to be actively controlled using said representative device.

13. The system of claim 1 wherein said display is configured to provide a live stream video of a representative supporting said medical procedure.

14. The system of claim 1 further comprising a second display, the second display configured to provide a live stream video of a representative supporting said medical procedure.

15. The system of claim 1 wherein said display is configured to provide a live stream video of at least one of a representative supporting said medical procedure, the visual data being received by the representative device, and a picture-in-picture view of the representative supporting said medical procedure and the visual data being received by the representative device.

16. The system of claim 1 wherein the support audio and visual data includes an avatar representation of a medical tool on an image or video, the image or video being created using said audio and visual data.

17. The system of claim 1 wherein said video camera is configured to provide one or more measurements of objects imaged by the video camera so that the one or more measurements may be shared with a representative supporting said medical procedure, medical personnel participating in said medical procedure, or both.

18. The system of claim 1 wherein said audio and visual data includes calibrated visual data suitable to indicate one or more measurements to a representative supporting the medical procedure and to allow the representative to project a proportioned avatar representation on the visual data.

19. The system of claim 1 wherein at least one of said display and a second display is configured for use as a whiteboard so that a representative supporting said medical procedure may remotely draw on said display or the second display.

20. The system of claim 19 wherein said display or said second display is configured such that medical personnel present in the operating room may manually draw on said display or the second display.

21. The system of claim 1 further comprising the representative device, the representative device being configured to display one or more assets from an asset library so as to provide a virtual reality depiction of one or more medical devices on an image or video created from said audio and visual data.

22. The system of claim 1 wherein the support audio or visual data comprises an image or video depicting a virtual representation of medical equipment created from said audio and visual data.

23. The system of claim 22 wherein said image or video includes a representation of a 3-dimensional object whose position is defined independent of a position or orientation of the video camera.

24. The system of claim 22, wherein the system is configured to automatically save a record of a virtual representation of one or more medical devices used during the medical procedure.

25. The system of claim 22, wherein the system is configured to record a video of the medical procedure and record a virtual representation of one or more medical devices used during the medical procedure.

26. The system of claim 1 further comprising said representative device, the representative device including a pen or stylus configured to provide mark ups or notes on a captured image or video selected from said audio and visual data.

27. The system of claim 1 wherein the one or more communication devices include at least one computer processor programmed for running in a guided access mode and creating a file documenting the medical procedure.

28. The system of claim 1 wherein the one or more communication devices comprise a computer device configured to automatically identify nearby candidate representative devices that are in physical proximity to the one or more communication devices and display usernames associated with said nearby candidate representative devices.

29. The system of claim 28 wherein the computer device is further configured to send an invitation code to one or more other candidate representative devices.

30. The system of claim 1 wherein the one or more communication devices comprise a computer device configured for accessing a registry of qualified representatives and sending an invitation request to one or more candidate representative devices associated with one or more of said qualified representatives.

31-70. (canceled)
